(19) United States
(12) Patent Application Publication
(10) Pub. No.: US 2024/0335104 A1
(43) Pub. Date: Oct. 10, 2024

(54) Visual Field Testing Apparatus

(71) Applicant: FINDEX Inc., Tokyo (JP)

(72) Inventor: Teruo AIBARA, Tokyo (JP)

(21) Appl. No.: 18/743,015

(22) Filed: Jun. 13, 2024

Related U.S. Application Data
(63) Continuation-in-part of application No. PCT/JP2022/041472, filed on Nov. 8, 2022.
Application No. PCT/JP2022/041472 is a continuation-in-part of application No. PCT/JP2021/033444, filed on Sep. 13, 2021.
Application No. PCT/JP2021/033444 is a continuation-in-part of application No. PCT/JP2021/025184, filed on Jul. 2, 2021.
Application No. PCT/JP2021/025184 is a continuation-in-part of application No. PCT/JP2021/016860, filed on Apr. 27, 2021.
(63) Continuation-in-part of application No. 18/305,325, filed on Apr. 22, 2023.
Application No. 18/305,325 is a continuation-in-part of application No. 18/173,724, filed on Feb. 23, 2023.
Application No. 18/173,724 is a continuation-in-part of application No. 17/937,780, filed on Oct. 4, 2022.
Application No. 17/937,780 is a continuation-in-part of application No. PCT/JP2021/016860, filed on Apr. 27, 2021.
(63) Continuation-in-part of application No. 18/173,724, filed on Feb. 23, 2023.
(63) Continuation-in-part of application No. 17/937,780, filed on Oct. 4, 2022.

(30) Foreign Application Priority Data
Jun. 16, 2020 (JP) 2020-103472
Aug. 25, 2020 (JP) 2020-141312
Nov. 5, 2020 (JP) 2020-184793
Dec. 24, 2021 (JP) 2021-210070

(51) Int. Cl. A61B 3/024; A61B 3/00; A61B 3/113

(52) U.S. Cl. CPC A61B 3/024; A61B 3/0091; A61B 3/113

(57) ABSTRACT

A visual field testing apparatus comprises a display for displaying targets; a line of sight detection unit that detects a line of sight of a user and that outputs line of sight information pertaining to a direction of the line of sight; a visual recognition determination unit that carries out determination as to whether or not the user has visually recognized a target based on line of sight information and position information of the target; and a visual field testing unit that sequentially displays targets at prescribed locations at the display and that carries out testing of a range of visual field based on output from the visual recognition determination unit; wherein the visual field testing unit comprises a visual recognition detection feedback unit which, when it is detected due to output from the visual recognition determination unit that the user has visually recognized a target which is currently displayed at the display, carries out feedback processing for a prescribed time to let the user know that detection has occurred.

### CROSS-REFERENCE TO RELATED APPLICATION(S), BENEFIT/PRIORITY CLAIM(S), AND INCORPORATION(S) BY REFERENCE

This application is a continuation-in-part of and claims benefit under 35 USC 120 and 365(c) to copending International Application No PCT/JP2022/041472, entitled “Visual Field Testing Apparatus”, filed 8 Nov. 2022; further is a continuation-in-part of and claims benefit under 35 USC 120 and 365(c) to International Application No PCT/JP2021/033444, entitled “Characteristic Inspection System”, filed 13 Sep. 2021, the aforesaid PCT/JP2021/033444 having been copending at the time of filing of the aforesaid PCT/JP2022/041472; further is a continuation-in-part of and claims benefit under 35 USC 120 and 365(c) to International Application No PCT/JP2021/025184, entitled “Visibility Determination System and Visual Field Test Apparatus”, filed 2 Jul. 2021, the aforesaid PCT/JP2021/025184 having been copending at the time of filing of the aforesaid PCT/JP2021/033444; further is a continuation-in-part of and claims benefit under 35 USC 120 and 365(c) to International Application No PCT/JP2021/016860, entitled “Visual Field Testing Apparatus”, filed 27 Apr. 2021, the aforesaid International Application No PCT/JP2021/016860 having been copending at the time of filing of the aforesaid International Application No PCT/JP2021/025184; further is a continuation-in-part of and claims benefit under 35 USC 120 to copending U.S. patent application Ser. No. 18/305,325, entitled “Characteristic Inspection System”, filed 22 Apr. 2023, which is a continuation-in-part of and claims benefit under 35 USC 120 to copending U.S. patent application Ser. No. 18/173,724, entitled “Visibility Determination System and Visual Field Test Apparatus”, filed 23 Feb. 2023, which is a continuation-in-part of and claims benefit under 35 USC 120 to copending U.S. patent application Ser. No. 17/937,780, entitled “Visual Field Testing Apparatus”, filed 4 Oct. 2022, which is a continuation-in-part of and claims benefit under 35 USC 120 and 365(c) to the aforesaid International Application No PCT/JP2021/016860, the aforesaid U.S. patent application Ser. No. 17/937,780 having been copending at the time of filing of the aforesaid U.S. patent application Ser. No. 18/173,724, and the aforesaid International Application No PCT/JP2021/016860 having been copending at the time of filing of the aforesaid U.S. patent application Ser. No. 17/937,780; further is a continuation-in-part of and claims benefit under 35 USC 120 to copending U.S. patent application Ser. No. 18/173,724, entitled “Visibility Determination System and Visual Field Test Apparatus”, filed 23 Feb. 2023, which is a continuation-in-part of and claims benefit under 35 USC 120 to copending U.S. patent application Ser. No. 17/937,780, entitled “Visual Field Testing Apparatus”, filed 4 Oct. 2022, which is a continuation-in-part of and claims benefit under 35 USC 120 and 365(c) to the aforesaid International Application No PCT/JP2021/016860, the aforesaid U.S. patent application Ser. No. 17/937,780 having been copending at the time of filing of the aforesaid U.S. patent application Ser. No. 18/173,724, and the aforesaid International Application No PCT/JP2021/016860 having been copending at the time of filing of the aforesaid U.S. patent application Ser. No. 17/937,780; further is a continuation-in-part of and claims benefit under 35 USC 120 to the aforesaid U.S. patent application Ser. No. 17/937,780, which is a continuation-in-part of and claims benefit under 35 USC 120 and 365(c) to the aforesaid International Application No PCT/JP2021/016860, the aforesaid International Application No PCT/JP2021/016860 having been copending at the time of filing of the aforesaid U.S. patent application Ser. No. 17/937,780; further claims benefit of priority under 35 USC 119(a)-(d) to Japanese Patent Application No 2021-210070, entitled “Visual Field Testing Apparatus”, filed 24 Dec. 2021; further claims benefit of priority under 35 USC 119(a)-(d) to Japanese Patent Application No 2020-184793, entitled “Characteristic Inspection System”, filed 5 Nov. 2020; further claims benefit of priority under 35 USC 119(a)-(d) to Japanese Patent Application No 2020-141312, entitled “Visibility Determination System and Visual Field Test Apparatus”, filed 25 Aug. 2020; and further claims benefit of priority under 35 USC 119(a)-(d) to Japanese Patent Application No 2020-103472, entitled “Visual Field Testing Apparatus”, filed 16 Jun. 2020, the contents of all of which applications are incorporated herein in their entireties by reference.

### STATEMENT REGARDING PRIOR DISCLOSURES BY INVENTOR UNDER 37 CFR 1.77(b)(6)

The following patents and published patent applications are disclosures by, originating with, and/or attributable to the inventor of the present application:
WO 2021 256100 A1 (“Visual Field Testing Apparatus”; 23 Dec. 2021);
JP 2021 194289 A (“Visual Field Testing Apparatus”; 27 Dec. 2021);
WO 2022 044548 A1 (“Visibility Determination System and Visual Field Test Apparatus”; 3 Mar. 2022);
JP 2022 037273 A (“Visibility Determination System and Visual Field Test Apparatus”; 9 Mar. 2022);
WO 2022 097368 A1 (“Characteristic Inspection System”; 12 May 2022);
JP 2022 074616 A (“Characteristic Inspection System”; 18 May 2022);
US 2023 0023605 A1 (“Visual Field Testing Apparatus”; 26 Jan. 2023);
US 2023 0190091 A1 (“Visibility Determination System and Visual Field Test Apparatus”; 22 Jun. 2023);
WO 2023 119913 A1 (“Visual Field Testing Apparatus”; 29 Jun. 2023);
JP 2023 094652 A (“Visual Field Testing Apparatus”; 6 Jul. 2023); and
US 2023 0255477 A1 (“Characteristic Inspection System”; 17 Aug. 2023).

### FIELD OF THE INVENTION

The present invention relates to a visual field testing apparatus.

### BACKGROUND

At a visual field testing apparatus for testing visual field in accordance with one embodiment of the present invention, a user might be made to use a button or other such input apparatus to input whether or not a target has been seen by his or her eye and recognized.

However, depending on the user, there have been cases in which the user is unfamiliar with operation of the input apparatus, or due to tension or the like is unable to skillfully operate the input apparatus, and so fails to properly carry out input therewith, as a result of which there has been inability to accurately carry out visual field testing.

At a visual field testing apparatus for testing visual field in accordance with another embodiment of the present invention, the line of sight which is the direction in which a user is looking might be automatically detected by a line of sight detection apparatus, and determination might be automatically made as to whether or not the user visually recognizes the target.

In accordance with some embodiments of the present invention, visual field testing might take 10 minutes or more. And where visual recognition determination is carried out automatically, it will in many cases be necessary for the user who is the test subject to constantly be made to gaze continuously at targets that are sequentially displayed at a display during this period.

Where this is the case, the user may be continuously made to experience a constant state of stress during visual field testing, and the burden placed on the user in connection with testing may be extremely large. For this reason, among children and the elderly, for whom it may be difficult to maintain concentration for long periods of time, there are cases in which gazing, which was supposed to have been for a prescribed amount of time, has been discontinued partway through, preventing accurate testing from being carried out.

There is therefore a need to provide a visual field testing apparatus such as will reduce the burden placed on the user in accompaniment to visual field testing and permit more accurate visual field testing to be carried out.

### SUMMARY OF INVENTION

A visual field testing apparatus associated with one embodiment of the present invention for testing a range of visual field of a user may comprise display(s) for displaying target(s).

Such a visual field testing apparatus may further comprise a line of sight detection unit that detects a line of sight of the user and that outputs line of sight information pertaining to a direction of the line of sight.

Such a visual field testing apparatus may further comprise a visual recognition determination unit that carries out determination as to whether or not the user has visually recognized the targets based on the line of sight information and position information of target(s).

Such a visual field testing apparatus may further comprise a visual field testing unit that sequentially displays targets at prescribed locations at display(s) and that carries out testing of the range of visual field based on output from the visual recognition determination unit.

In some embodiments, the visual field testing unit may comprise a visual recognition detection feedback unit. When it is detected due to the output from the visual recognition determination unit that the user has visually recognized the target which is currently displayed at the display, the visual recognition detection feedback unit may carry out feedback processing for a prescribed time to let the user know that detection has occurred.

By providing feedback with respect to detection of visual recognition of target(s) by a user, at least some embodiments of the present invention may allow the user to be released even for a short time from the state of stress, may make it possible for the user to better concentrate and gaze at target(s), and may permit more accurate visual field testing to be carried out.

Other embodiments, systems, methods, and features, and advantages of the present invention will be or become apparent to one with skill in the art upon examination of the following drawings and detailed description. It is intended that all such additional systems, methods, features, and advantages be included within this description, be within the scope of the present invention, and be protected by the accompanying claims.

### BRIEF DESCRIPTION OF DRAWINGS

Many aspects of the invention can be better understood with reference to the following drawings. The components in the drawings are not necessarily to scale, emphasis instead being placed upon clearly illustrating the principles of the present invention. Moreover, in the drawings, like reference numerals designate corresponding parts throughout the several views.

FIG. 1 is a schematic diagram showing in simplified fashion the constitution of a visual field testing apparatus associated with an embodiment of the present invention.

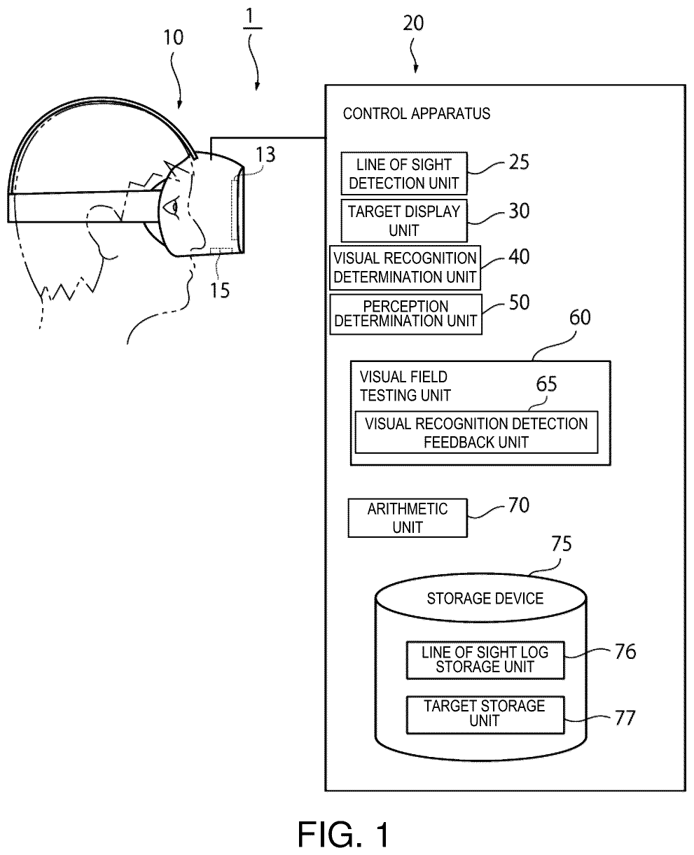

FIG. 2 is a drawing for describing visual recognition detection feedback processing associated with an embodiment of the present invention.

FIG. 3 is a drawing for describing a variation on visual recognition detection feedback processing associated with an embodiment of the present invention.

FIG. 4 is a drawing for describing a variation on visual recognition detection feedback processing associated with an embodiment of the present invention.

### EMBODIMENTS FOR CARRYING OUT INVENTION

Except where otherwise clear from context, note that use of the singular or plural herein should not be understood in a limiting or exclusionary sense where this would constitute an unreasonable interpretation of the present invention. For example, depending on the embodiment in question, it may be that both eyes are tested during a round of testing but only one eye is actively undergoing testing at any given moment. Likewise, depending on the embodiment in question, it may be that multiple sets of gazing targets and measurement targets are displayed during a round of testing but only one set, and more particularly only either the gazing target or the measurement target of that set, is actively being displayed at any given moment. Furthermore, depending on the embodiment in question, it may be that both eyes are tested but only one eye is being described by way of example. Moreover, independent displays may, for example, be used for the right eye and the left eye, or a single large display that has been divided into a region for the right eye and a region for the left eye may be used, and depending on the embodiment in question the various units and other components for testing of the right eye and the left eye may similarly take the form of a single component for both eyes or separate components for each of the respective eyes. It should therefore be noted that regardless of whether the present specification makes reference in the singular, for example, to display, lens, camera, mirror, emitter, and so forth, or conversely makes reference in the plural, for example, to displays, lenses, cameras, mirrors, emitters, and so forth, such references should except where otherwise clear from context be understood in a collective or nonexclusionary sense, without limitation as to whether there is a single such component for both eyes or separate components for the respective eyes. Similarly, references made herein to gazing target(s), measurement target(s), and the various apparatus(es) and unit(s) and so forth should, except where otherwise clear from context, be understood as nonlimiting with respect to number.

Visual field testing apparatus 1, which is associated with an embodiment of the present invention, will be described in detail hereinbelow with reference to the drawings. In accordance with the present embodiment, when a user who is a test subject actually sees and visually recognizes targets that are sequentially displayed at display 13 during visual field testing, feedback from the apparatus lets the user know that said visual recognition has been detected.

Visual field testing apparatus 1 in accordance with the present embodiment may comprise head mounted display (HMD) 10, and control apparatus 20 which may be a computer connected by a communication cable to HMD 10. HMD 10, which may be equipment that is mounted on the head of the user during visual field testing, might comprise display 13 which displays targets, camera 15 for capturing images of the eyes of the user, and so forth.

HMD 10 may be equipped with line of sight detection functionality by which a line of sight that is a direction in which the user on whom HMD 10 is mounted is looking with his or her eye(s) is automatically detected in cooperation with line of sight detection unit 25, described below. Display 13 and camera 15 may respectively have units installed thereat for the right eye and the left eye.

Control apparatus 20 may comprise central processing unit(s) (CPU) or other such arithmetic unit(s) 70 for carrying out various types of operations, as well as hard disc drive(s) (HDD) for storing various types of information, and/or random access memory or memories (RAM) capable of being used as a work area during arithmetic processing, or other such storage device(s) 75.

Storage device 75 may comprise line of sight log storage unit 76 which sequentially records line of sight information detected by line of sight detection unit 25, described below; and may further comprise target storage unit 77 which records target information pertaining to targets to be displayed at display 13.

Furthermore, control apparatus 20 might comprise-in terms of functionalities-line of sight detection unit 25, target display unit 30, visual recognition determination unit 40, perception (visibility) determination unit 50, and/or visual field testing unit 60, these functionalities being implemented by causing prescribed program(s) stored at storage device 75 to be executed by arithmetic unit 70, for example.

Line of sight detection unit 25 might detect the direction in which the user is looking, i.e., the line of sight, based on captured image(s) of the eye(s) of the user as output by camera 15. More specifically, line of sight detection unit 25 might output two angular components (theta, phi) which are polar coordinates (spherical coordinates) in the form of line of sight information (line of sight angles) indicating the direction of the line of sight. In a preferred embodiment, this line of sight detection is carried out independently for the right eye and the left eye.

Target display unit 30 may cause white targets of prescribed size to be sequentially displayed at prescribed sensitivity (brightness) at prescribed coordinate locations at display 13 based on target information recorded at target storage unit 77.

Visual recognition determination unit 40 may carry out determination as to whether or not the target has actually been seen and visually recognized by the user, based on the line of sight detected by line of sight detection unit 25 and the coordinates of the target displayed at display 13. More specifically, visual recognition determination might be carried out based on whether or not an imaginary line that is an extension in the direction of the line of sight detected by line of sight detection unit 25 within a prescribed coordinate system would physically collide with the target in question. In accordance with the present embodiment, a determination will be made that there has been a collision and that visual recognition has taken place if upon comparison of the line of sight angle detected by line of sight detection unit 25 and the target angle indicating the location of the target in question, the angular difference therebetween is within 2 degrees.

Note that it is also possible for visual recognition determination unit 40 to cause visual recognition determination to be carried out using logical determinative techniques that make use of logged line of sight information. For example, a constitution may be adopted in which visual recognition determination unit 40 causes logical determination of visual recognition to be carried out when the line of sight approaches the target in question and continues to be located near said target.

Perception determination unit 50 may carry out determination as to whether or not a target at a position other than a position which is in the line of sight of the user has been seen by (has caught the eye of) the user, i.e., whether or not it has been perceived. Perception determination unit 50 might, for example, be such that after the target has been displayed at display 13, determination as to whether perception occurred at the time that the target was displayed is carried out based on the arrival path which is the path taken by the line of sight, and/or the arrival time which is the time taken by the line of sight, in going from the position that the line of sight was in at the time that display occurred until it arrives (is visually perceived) at said target.

Visual field testing unit 60 may control line of sight detection unit 25, target display unit 30, visual recognition determination unit 40, and perception determination unit 50, and may cause visual field testing to be carried out. Visual field testing unit 60 may be equipped with visual recognition detection feedback unit 65 which, when it is detected due to output from visual recognition determination unit 40 that the user has visually recognized a target displayed at display 13 during visual field testing, lets the user know that visual recognition has been detected.

Visual recognition detection feedback unit 65 may control target display unit 30, and may carry out processing to let the user know that detection of visual recognition has occurred by changing the color of the target displayed at display 13 from the standard white color to another color.

In accordance with the present embodiment, visual recognition detection feedback unit 65 might carry out feedback processing by, 500 ms after detection of visual recognition has occurred, changing the color of the target to green for a period of 300 ms. Together with the conclusion of feedback processing, the target that was visually recognized is—while still green—made to disappear, and the next target is promptly displayed in the standard white color at display 13.

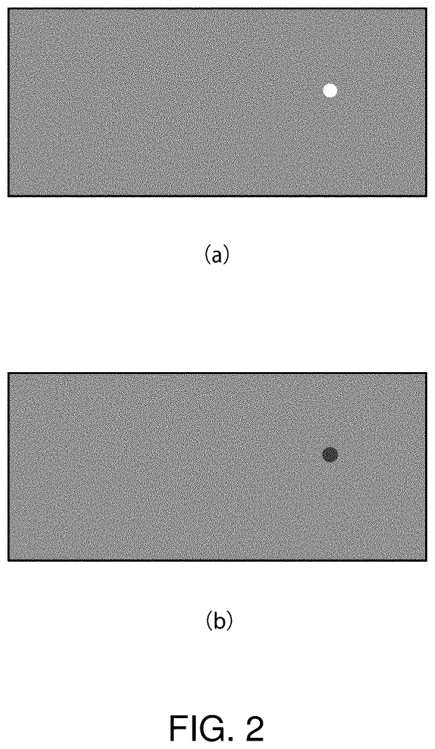

(a) at FIG. 2 shows a situation in which a target is displayed in the standard white color at display 13; (b) at FIG. 2 shows a situation in which a target is displayed in green at display 13.

How much time should elapse after detection of visual recognition has occurred before feedback processing is carried out, the period of time during which feedback processing should be carried out, the timing with which a target that has been visually recognized should be made to disappear, the timing with which the next target should be displayed, and so forth may of course be varied as appropriate. Note further that, to maintain a time capable of being noticed by the user, while also not causing visual field testing time to be too long, it is preferred that the period of time during which feedback processing is carried out be 200 ms to 800 ms.

Furthermore, regarding the color to which the target is changed for feedback processing, this is not limited to green, it being possible to employ blue, red, or any other color as appropriate such as will readily allow the user to be made aware of the change in color.

During visual field testing, because the user who is the test subject is instructed to continue to gaze at the targets displayed at display 13, he or she may be continuously in a state of stress throughout testing, and so there are situations in which, when concentration is interrupted partway therethrough and it becomes impossible to continue to follow the targets, reduction in the accuracy with which testing is carried out can occur.

To address this, in accordance with the present embodiment, notwithstanding that it is only for an extremely short time until the next target is displayed, the fact that feedback is provided to let the user know that visual field testing apparatus 1 has detected that the user has visually recognized the target allows the user to be released from the state of stress, which makes it dramatically easier to maintain the concentration necessary for testing, as a result of which it is possible to further increase the accuracy of testing.

Furthermore, in accordance with the present embodiment, the change in color of the target is easily noticed by the user as he or she continues to look at the target; and furthermore, as this requires no addition of some member or the like, and it is possible to easily implement this through modification of program(s), this makes it possible for great benefit to be easily provided.

Above, description has been given with respect to visual field testing apparatus 1 associated with the present embodiment; in accordance with the present embodiment, by providing feedback to let the user know that visual field testing apparatus 1 has detected that the user has visually recognized the target, it is possible to cause visual field testing to be carried out with increased accuracy.

Description will next be given with respect to a variation on the processing by which visual recognition detection feedback is given to the user by visual recognition detection feedback unit 65. Whereas in the foregoing embodiment feedback was provided by changing the color of the target displayed at display 13 for a prescribed time, it is also for example possible to provide feedback by changing the shape of the target, or by changing the combination of both the color and shape of the target.

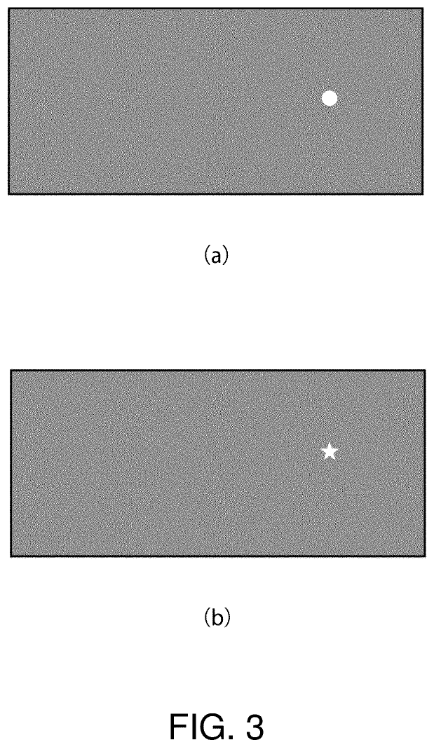

FIG. 3 is a drawing showing an example of how the shape of the target might be changed so as to provide feedback. (a) at FIG. 3 shows a situation in which a target is displayed with the standard circular shape at display 13; (b) at FIG. 3 shows a situation in which a target is displayed in the shape of a star at display 13. Thus, a target that would be standardly displayed with circular shape may be made to be in the shape of a star during the period when feedback is being provided. Feedback may of course be provided not only by causing the shape thereof to be changed to that of a star, but also by causing the shape thereof to be changed to that of a square or other such polygon or other such shape as appropriate.

Furthermore, as visual method(s) for providing feedback with respect to detection of visual recognition, besides changing the color and/or shape of the target, it is also possible to change the color of the background at display 13 and/or to cause a symbol or text indicating that detection has occurred to be displayed at prescribed location(s) at display 13.

Moreover, as method(s) for providing feedback with respect to detection of visual recognition, besides methods in which this is made known visually, it is also possible to make this known to the user tactilely, auditorily, and/or by way of other such sense(s). Where feedback is to be provided to the user tactilely, a small vibrating element might be mounted on HMD 10, and the vibrating element might be made to vibrate for a prescribed time (e.g., for 300 ms) while feedback is being provided.

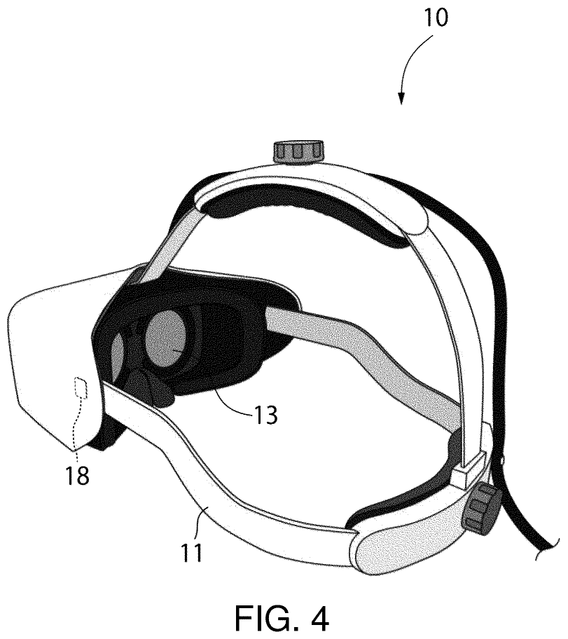

FIG. 4 is a schematic diagram showing in simplified fashion the constitution of HMD 10 in a situation in which tactile feedback is provided to the user. As shown in same drawing, by installing a small vibrating element 18 at the interior on either side of case 11 of HMD 10, it will be possible to provide the user with feedback without disturbing the way in which HMD 10 is mounted on the user.

Furthermore, where feedback is to be provided auditorily, by causing speaker(s), not shown, at visual field testing apparatus 1 to produce an audible notification, it will be possible to let the user know that visual recognition has been detected. It is of course also possible to carry out feedback processing by providing a combination of visual, tactile, auditory, and/or other such feedback.

While embodiments of the present invention have been described above, embodiments for carrying out the present invention are not limited to the foregoing embodiments, a great many variations being possible without departing from the gist of the present invention. For example, whereas an HMD that is mounted on the head was employed as display, it is also possible to employ an ordinary liquid crystal monitor of the standalone or wall-mounted type or the like.

### EXPLANATION OF REFERENCE NUMERALS

1 Visual field testing apparatus
10 HMD
11 Case
13 Display
15 Camera
18 Vibrating element
20 Control apparatus
25 Line of sight detection unit
30 Target display unit
40 Visual recognition determination unit
50 Perception determination unit
60 Visual field testing unit
65 Visual recognition detection feedback unit
70 Arithmetic unit
75 Storage device
76 Line of sight log storage unit
77 Target storage unit

What is claimed is:

1. A visual field testing apparatus for testing a range of visual field of a user, the visual field testing apparatus comprising:
a display for displaying a first target;
a line of sight detection unit that detects a line of sight of the user and that outputs line of sight information pertaining to a direction of the line of sight;
a visual recognition determination unit that carries out determination as to whether or not the user has visually recognized the first target based on the line of sight information and position information of the first target; and
a visual field testing unit that displays the first target at a first prescribed location at the display;
wherein the first target is one among a plurality of targets and the first prescribed location is one among a plurality of prescribed locations, and the visual field testing unit sequentially displays the plurality of targets at the plurality of prescribed locations and carries out testing of the range of visual field based on output from the visual recognition determination unit;
wherein the visual field testing unit comprises a visual recognition detection feedback unit which, when it is detected due to the output from the visual recognition determination unit that the user has visually recognized the first target when the first target is currently displayed at the display, carries out feedback processing for a prescribed time to let the user know that detection has occurred.

2. The visual field testing apparatus according to claim 1 wherein the visual recognition detection feedback unit is such that the feedback processing takes a form of causing the first target to change color when the first target is currently displayed at the display and has been visually recognized.

3. The visual field testing apparatus according to claim 1 wherein the visual recognition detection feedback unit is such that the feedback processing takes a form of causing the first target to change shape when the first target is currently displayed at the display and has been visually recognized.

4. The visual field testing apparatus according to claim 2 wherein the visual recognition detection feedback unit is such that the feedback processing takes a form of causing the first target to change shape when the first target is currently displayed at the display and has been visually recognized.

5. The visual field testing apparatus according to claim 1 wherein the visual field testing unit is such that, upon conclusion of the feedback processing, the first target is made to disappear after the first target has been visually recognized, and a second target is thereafter displayed at the display.

6. The visual field testing apparatus according to claim 2 wherein the visual field testing unit is such that, upon conclusion of the feedback processing, the first target is made to disappear after the first target has been visually recognized, and a second target is thereafter displayed at the display.

7. The visual field testing apparatus according to claim 3 wherein the visual field testing unit is such that, upon conclusion of the feedback processing, the first target is made to disappear after the first target has been visually recognized, and a second target is thereafter displayed at the display.

8. The visual field testing apparatus according to claim 4 wherein the visual field testing unit is such that, upon conclusion of the feedback processing, the first target is made to disappear after the first target has been visually recognized, and a second target is thereafter displayed at the display.

9. The visual field testing apparatus according to claim 1 wherein the prescribed time during which the feedback processing is carried out is between 200 ms and 800 ms.

10. The visual field testing apparatus according to claim 2 wherein the prescribed time during which the feedback processing is carried out is between 200 ms and 800 ms.

11. The visual field testing apparatus according to claim 3 wherein the prescribed time during which the feedback processing is carried out is between 200 ms and 800 ms.

12. The visual field testing apparatus according to claim 4 wherein the prescribed time during which the feedback processing is carried out is between 200 ms and 800 ms.

13. The visual field testing apparatus according to claim 5 wherein the prescribed time during which the feedback processing is carried out is between 200 ms and 800 ms.

14. The visual field testing apparatus according to claim 6 wherein the prescribed time during which the feedback processing is carried out is between 200 ms and 800 ms.

15. The visual field testing apparatus according to claim 7 wherein the prescribed time during which the feedback processing is carried out is between 200 ms and 800 ms.

16. The visual field testing apparatus according to claim 8 wherein the prescribed time during which the feedback processing is carried out is between 200 ms and 800 ms.
